(19) United States
(12) Patent Application Publication
(10) Pub. No.: US 2026/0106042 A1
(43) Pub. Date: Apr. 16, 2026

(54) ARTIFICIAL-INTELLIGENCE TECHNIQUES FOR NEUROTOXICITY DETECTION

(71) Applicant: NEUROVIGIL, INC., Moffett Field, CA (US)

(72) Inventor: Philip LOW, Moffett Field, CA (US)

(73) Assignee: NEUROVIGIL, INC., Moffett Field, CA (US)

(21) Appl. No.: 19/420,482

(22) Filed: Dec. 15, 2025

Related U.S. Application Data
(63) Continuation of application No. PCT/US2024/038103, filed on Jul. 15, 2024.
(60) Provisional application No. 63/527,011, filed on Jul. 15, 2023.

(51) Int. Cl. G16H 50/30; A61B 5/00

(52) U.S. Cl. CPC G16H 50/30; A61B 5/4064; A61B 5/4848

(57) ABSTRACT

Detecting neurotoxicity associated abnormalities due to certain medication therapies can be significant for identifying various neurological disorders. The present disclosure relates to detection of neurotoxicity related disorders by leveraging an interface including one or more sets of queries and a set of components to receive a set of responses corresponding to the sets of queries. The techniques, as disclosed herein, may use one or more attributes of the set of responses including consistency, complexity, grammar and spelling correctness, and time taken during the responses. The disclosed technique may preprocess the responses, extract features and generate one or more metrics corresponding to the one or more attributes of the one or more responses provided by the subject during a session with the interface. The generated metrics over a baseline session and subsequent sessions may be used to determine trends in the metrics which may be used to estimate the extent of neurotoxicity developed by a subject and may trigger an alert about a potential neurotoxicity or a preventative measure to reduce a likelihood of further neurotoxicity.

### CROSS-REFERENCE TO RELATED APPLICATIONS

This application is a continuation of International Patent Application No. PCT/US2024/038103, filed on Jul. 15, 2024, which claims priority to U.S. Provisional Patent Application No. 63/527,011, filed on Jul. 15, 2023. The entire disclosures of the aforementioned applications are incorporated by reference herein in their entireties for all purposes.

### BACKGROUND

Different medication therapies may have significantly high neurotoxicity risks because of harmful chemicals or toxic agents contained in them. For example, CAR T-Cell therapy may be administered to subjects with different types of liquid tumors, such as lymphoma, leukemia, and multiple myeloma. For example, some randomized controlled trials (RCTs) that included subjects who had received this chemotherapy treatment found that 25% of the subjects experienced neurotoxicity, and 8% of the subjects experienced severe neurotoxicity. Neurotoxicity results in damaging brain or the peripheral nervous system. Sustained neurotoxicity results in death of nerve cells, resulting in an irreversible damage to the brain leading to symptoms such as cognitive impairment, motor impairment, or sensory impairment etc. Consequently, subjects may suffer from mental illness such as anxiety, confusion, depression to name a few.

The first line of “treatment” for neurotoxicity is to detect the neurotoxicity, and subsequently reduce or stop the exposure to a toxic agent (or harmful chemical) causing the neurotoxicity. Unfortunately, due to a broad range of symptoms that may be associated with neurotoxicity, its early and reliable detection still remains a significant challenge.

### SUMMARY

Certain aspects and features of the present disclosure relate to identification of neurotoxicity via analysis of one or more responses to one or more queries obtained through an interface on a user device. The interface on the user device may present a set of queries and a set of components configured to receive a set of responses from the user corresponding to the set of queries. A particular set of responses corresponding to the set of queries may be received from the user device at a backend or a server for analysis. The particular set of responses may be processed using one or more artificial intelligence techniques to generate one or more metrics. Each of at least one of the one or more metrics may be based on: an extent to which responses provided by a user in a given session with the interface are consistent with each other or with one or more responses provided by the user in one or more other sessions; a complexity or sophistication of responses provided by the user in the given session with the interface; a degree to which responses provided by the user in the given session with the interface accord with grammatical rules and/or proper spelling; one or more amounts of times that the user spent providing responses during the given session; one or more amounts of times that the user paused while providing two consecutive responses during the given session; and a cumulative amount of time that the user paused while providing responses during the given session.

The consistency between one or more responses of the set of responses may be estimated by generating one or more distributions of positions in a multidimensional space corresponding to one or more tokens in each of the one or more responses and performing a comparison between distributions corresponding to the one or more responses. The consistency may be represented as a metric (e.g., a numeric metric) generated by using, for example, similarity measures, to estimate consistency between the responses of the set of responses based on the comparison.

In some instances, a metric may represent the complexity or sophistication of one or more responses corresponding to a query. For example, one or more large language models (LLM) may be used to generate a metric that represents a complexity of the response.

A metric related to time response of a subject may be generated using a delay between presentation of one or more queries and receipt of one or more corresponding responses. For example, a delta period may be calculated for each query that is set to be equal to a time between when the query was presented and a response was provided. The metric related to the time response may be defined to be or may relate to a statistic based on multiple delta periods (e.g., a mean, median, mode, etc.). As another example, the metric may be defined to be or may relate to a time between an initial presentation of at least part of a set of queries and receipt of responses to all of the queries. It will be appreciated that the metric may be transformed and/or normalized based on past metrics and/or data associated with the subject and/or other metrics associated with other subjects.

A metric may indicate or may be based on a duration of a session or a cumulative time for which it is was estimated that the user was actively involved in a session. For example, the cumulative time may account for all times during which an app or webpage that presented the queries was in view (e.g., as opposed to another app or webpage obscuring some or all of the query app or webpage and/or as opposed to a device that is or was presenting the app or webpage being asleep).

A metric may be based on how frequently pauses are detected while input corresponding to each of one or more individual responses are received, how frequently pauses are detected between responses, and/or durations of one or both types of pauses. For example, for a given response, each time window across a duration of the response input may be characterized as “active input” (e.g., when a user is typing part of the response) or “pause” (when no such input is received). For each response, a percentage of time windows assigned to the “pause” category can be calculated, and the metric may be defined as a statistic generated based on the percentages associated with multiple queries (e.g., a mean, median or mode percentage).

A composite score can be generated based on the one or more metrics. The composite score may include a statistic generated based on multiple metrics of the one or more metrics. For example, the composite score may be defined to be or may be defined based on a mean, median, mode of the metrics. In some instances, the composite score is based on one or more relative metrics. For example, each of at least one of the one or more metrics may be normalized based on other metrics of a population (e.g., a healthy population, a population that has or is to receive a given medication or medication of a given type, a population exhibiting a given symptom, etc.). As another example, a relative metric may include a difference between a metric corresponding to a recent session (or session set) for a subject relative to that from one or more past sessions. Such a relative metric may include a derivative, second derivative etc.

In some instances, the composite score may be generated in a manner such that metrics (or values calculated thereon) are weighted. As one illustration, during an initial session for a subject, a weight may be assigned to each metric based on how the metric for the subject compares to that of a population (e.g., where a weight may be defined such that it is higher when the metric is associated an upper end of the metric distribution in the population, or the reverse). As another illustration, a weight may be dynamically assigned to a metric based on a degree to which the metric has changed across recent sessions (e.g., with a higher weight assigned to a metric when it has exhibited a change higher relative to other metrics for the subject across recent sessions). As yet another illustration, a weight may be assigned based on population data (e.g., that indicates a degree to which various metrics change across sessions generally or for a sub-population).

One or more scores corresponding to the one or more metrics may be used to estimate the level of neurotoxicity by accumulating scores from two or more sessions and determining whether the scores or the composite score satisfy a condition of three conditions corresponding to negligible neurotoxicity, non-severe neurotoxicity, or severe neurotoxicity. In response to determining that the condition is satisfied, a presentation, transmission or action may be triggered that corresponds to an alert about a potential neurotoxicity or a preventative measure that is predicted to reduce a likelihood of further neurotoxicity. The condition may be configured to be satisfied when (for example) a composite score crosses a threshold, change in a composite score crosses a threshold, a slope of a composite score crosses a threshold, a metric crosses a threshold, a slope of a metric crosses a threshold, and/or any combination thereof. In some instances, an outlier-detection technique is performed to predict when a given metric or a given composite score is predicted to be an outlier, and use of any such metric or composite score is omitted for further processing (e.g., such that the given metric is not to contribute to a corresponding composite score or such that the given composite score is not to be evaluated using the condition). Each of one or more thresholds included in a condition may be an absolute or relative threshold. For example, a relative threshold may be defined as a specific percentage of a composite score or metric associated with a prior session and the subject or a statistic based thereupon. As another example, a relative threshold may be defined based on a set of other composite scores and/or a set of other metrics associated with a set of other subjects. It will be

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer program product tangibly embodied in a non-transitory machine-readable storage medium, including instructions configured to cause one or more data processors to perform part or all of one or more methods or processes disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

Various embodiments are described hereinafter with reference to the figures. It should be noted that the figures are not drawn to scale and that the elements of similar structures or functions are represented by like reference numerals throughout the figures. It should also be noted that the figures are only intended to facilitate the description of the embodiments. They are not intended as an exhaustive description of the disclosure or as a limitation on the scope of the disclosure.

FIG. 1 shows an example distribution of subjects developing neurotoxicity as a side effect of different medication therapies received by the subjects.

FIG. 2 shows one or more examples of different kinds of impairments experienced by the subjects due to neurotoxicity.

FIG. 3 shows an exemplary interface of a neurotoxicity detector on a user device for performing neurotoxicity assessments for a subject by asking a plurality of questions and recording and assessing characteristics of respective responses.

FIG. 4 illustrates various types of analyses performed on the respective responses of the subject, received from the interface of the user device of the subject.

FIG. 5 illustrates an exemplary preprocessing pipeline for the respective responses received from the user device of the subject.

FIG. 6 illustrates an example flow of a process for computing one or more composite scores associated with different metrics of the respective responses of the subject during a single session.

FIG. 7 is an example illustration of determining assessments for the respective responses in inter-session analysis based on the one or more scores associated with different metrics.

FIG. 8 shows an example graph of how a computed score of a metric across different sessions can be compared with a baseline score of the metric to estimate a level of neurotoxicity in a subject.

FIG. 9 shows an example illustration that how rate of change of the computed score of the metric across different sessions can be used to estimate the level of neurotoxicity in the subject.

FIG. 10 shows an example illustration of how inter-subject analyses of the one or more scores associated with one or more subjects can model population dynamic trends of scores to estimate the level of neurotoxicity in the subject.

FIG. 11 shows exemplary one or more actions of a management plan for a subject, depending on whether a given neurotoxicity condition is satisfied.

FIG. 12 illustrates an exemplary flow chart of a neurotoxicity detection process predicting neurotoxicity in a subject by analyzing the respective responses received from the user device of the subject.

FIG. 13 is an example illustration of a computer system in which one or more examples of a neurotoxicity estimator may be implemented.

### DETAILED DESCRIPTION

The present disclosure relates to new techniques, methods and systems for estimating a level of neurotoxicity in a subject by analyzing one or more responses received from the user interface of a user device of the subject. The user interface may be configured to present one or more queries to a user and receive respective responses from the user, store the queries and associated responses in user device or on a server storage in a cloud. The one or more queries can include open-ended questions configured to receive a response in the unstructured text of natural language. Thus, the interface may include for each query, a text based natural language response to the query, whereas a corresponding text box may be configured to allow a user to enter the text based natural language response to the query. Moreover, the interface may be configured to receive a dictated speech response, for a query, from the subject using a microphone of the user device, and the complete response may be stored in an audio file on the user device, and subsequently may be transferred to the server storage in the cloud. In the example, where the subject gave the response to a query by speaking into the microphone of the user device, the response may be analyzed using one or more speech processing techniques or artificial intelligence (AI) based speech processing techniques. In another example, the audio signal can be transcribed into text and the text may be analyzed for complexity or sophistication of responses using AI-based natural language processing in accordance with the response analysis method disclosed herein.

A response analysis method may include using one or more AI techniques to generate on or more scores and/or metrics to assess: (1) an extent to which responses provided by a user in a given session, using the interface on a user device, are consistent with each other; (2) complexity or sophistication of responses provided by a user in a given session using the interface on a user device; (3) a degree to which responses provided by a user in a given session, using the interface on a user device, accord with grammatical rules and/or proper spelling; (4) an amount of time that a user spent providing responses during a given session; (5) the number of times or cumulative amount of time that a user paused while providing response during a given session etc.

In some instances, a characteristic of a neurological signal may also or alternatively be used to generate a metric and/or score used in accordance with a technique disclosed herein. For example, one or more EEG signals may be collected during a session and/or during other time periods. The EEG signal(s) may be assessed to (for example) predict a degree of startle, concentration, effort, attention and/or confusion. For example, a signal (or portion thereof) may be transformed to detect one or more intensities within a beta band, which can then be used to estimate a degree of concentration, effort, attention and/or confusion; or a signal (or portion thereof) may be transformed to detect one or more intensities within a gamma band, which can then be used to estimate a degree of startle. The analysis may include quantifying a statistic pertaining to a signal strength in all or part of the beta band (e.g., a maximum, median, mode, mean, variance, standard deviation, or minimum) across part or all of a session. To illustrate, a variance statistic may be used to infer a degree to which a subject can hold their attention to a task. As another illustration, a median, mode, or mean statistic may be used to infer an overall level of startle. In some instances, a similar analysis pertaining to a neurological signal may be performed even outside of a session. This similar analysis may facilitate inferring general cognitive abilities (e.g., as to a maximum, median, mode or mean level of startle, concentration, effort, attention and/or confusion) and/or may provide data to normalize any statistics, variables, or data that correspond to one or more sessions. In these types of instances, the analysis may be performed using data that is not during a session but during which it is inferred that the subject is awake. In some instances, a similar or different analysis may be performed when it is inferred that the subject is asleep. To illustrate, EEG data, subject-input data and/or movement data may be used to infer that a subject is asleep. One or more features (e.g., associated with a gamma band) may be assessed to estimate a degree of startle, which may then be used (for example) to normalize a session metric and/or contribute to a score.

In some instances, a characteristic of data collected by one or more sensors that may also or alternatively be used to generate a metric and/or score used in accordance with a technique disclosed herein. The sensor may include (for example) a camera or a movement sensor, such as an accelerometer or gyrometer. A statistic may indicate a degree of movement during a session, shortly after a session (e.g., during a 10-second, 30-second, 1-minute, 5-minute, 10-minute or 30-minute interval afterwards) and/or outside a session. For example, a statistic may estimate a daily number of steps (where a score may be configured such that estimated fewer steps are correlated with an increased neurotoxicity probability). As another example, a statistic may predict a degree of tremors (where a score may be configured such that a prediction of stronger tremors is correlated with an increased neurotoxicity probability).

A score may be configured such that a likelihood of neurotoxicity is positively correlated with an estimated degree of concentration, effort, attention and/or confusion.

Additional or alternative assessments may characterize the degree to which each of one or more of the above-noted scores or metrics differ from the corresponding scores of metrics associated with one or more prior sessions of a user. In some examples, one or more of these assessment methods may include using the same AI models for computing scores for different metrics of each assessment or training an assessment specific AI model by factoring in assessment specific dynamics to compute scores for metrics of each assessment.

The one or more AI techniques may include using one or more trained machine learning models, which may include a generative model, a neural network, a long-short term memory model, a transformer model, a moving-average model (e.g., an autoregressive integrated moving average (ARIMA) model), a model that uses self-attention, such as ChatGPT, etc. In examples where two or more trained machine learning models are used to generate different scores of metrics, it is possible that the two or more trained machine learning models may be of the same type or of different types, and the example where the machine learning models are the neural networks they might be of the same network architecture or different network architectures as disclosed herein.

The AI techniques may include a preprocessing pipeline that may remove stopwords and/or transform various words or combination of words into corresponding tokens by using a library. The tokens may then be transformed into a vector using an encoding model, such as a term frequency-inverse document frequency TF-IDF vectorizer or a word2vec. The encoding model may be configured to perform the transformation based on the occurrence frequency of a given token (or a set of two or more related tokens) in a given response (or a combination of two or more responses), and/or what is the occurrence frequency of a given token or related tokens in an underlying set of responses, which may be generated based on one or more responses from two or more users. Tokens with a higher occurrence frequency in a set of responses for a user with a lower occurrence frequency in an underlying set of responses from two or more users may be interpreted as being relatively important for conveying the semantics or meaning of the underlying set. Moreover, the underlying training data set may be analyzed to identify pairs of tokens or a combination of three or more tokens thereof that have relatively higher frequency of occurrence in the same response, or a repones set of one or more responses.

Subsequently, a distance measure, showing semantic similarity of pairs of tokens or a combination of three or more tokens thereof, can be used to determine an extent to which responses provided by a user in a given session with the interface are consistent with each other or with one or more responses provided by the user in one or more other sessions, a complexity or sophistication of responses provided by the user in the given session with the interface, a degree to which responses provided by the user in the given session with the interface accord with grammatical rules and/or proper spelling, an amount of time that the user spent providing responses during the given session, or a number of times or cumulative amount of time that the user paused while providing responses during the given session.

In an example, a consistency between responses of a user to one or more queries during a single session or across the same response from one or more sessions can be estimated by comparing tokens in responses. For example, each token may be assigned to a position in a multi-dimensional space based on a baseline data set. A consistency of responses may be estimated based on an analysis of distribution of tokens in the multi-dimensional space: comparing a first distribution of positions, corresponding to the positions of tokens from a particular response or a particular session, to a second distribution of positions, corresponding to the positions of tokens from a different particular response or a different particular session; and calculating a statistic based on distances between representation of tokens (e.g., across responses in a single session or across responses from multiple sessions), etc. The statistic determines an extent to which responses provided by a user in a particular session with the interface are consistent with each other or with one or more responses provided by the user in one or more different particular sessions.

In an example, a generative model can be used to determine a consistency between responses to one or more queries during a single session or across the same response from one or more sessions of a subject. The generative model can be used to predict a response to one query based on a response from one or more other queries from the same session or one or more previous sessions of a subject. Stopwords may be removed from the predicted responses by the generative model and a true response from the user, and subsequently tokens can be generated, which can then be projected to different positions in a multi-dimensional space. A consistency of responses can be estimated based on a degree to which a distribution of tokens in the multidimensional space of the trues responses from the subject differs from a distribution of tokens in the multidimensional space of the corresponding predicted responses from the generative model. A consistency of responses can alternatively or additionally be estimated by computing a distance measure between projections of tokens in the multidimensional space of the true responses from the subject relative to projections of tokens in the multi-dimensional space of the corresponding predicted responses from the generative model.

In an example, one or more machine learning model or rule-based models may be used to determine an absolute or relative complexity or sophistication of one or more responses. For example, a model may be trained and configured to associate one or more tokens or a combination of tokens thereof with a complexity level. To illustrate, the model may be trained to associate tokens from a first subset of training data (e.g., scientific manuscripts) with higher complexity metrics than that of tokens from a second subset of training data (e.g., social media posts).

In an example, the model may be trained in an unsupervised fashion to learn how various features of content correspond with complexity. In another example, one or more rules may be defined to calculate a complexity or sophistication of one or more responses based on: a distribution of syllables per word, a distribution of words per sentence, how frequently various words or phrases are used in a baseline dataset of responses to queries, or the type(s) of punctuation that is used.

In an example, a machine-learning or rule-based model can estimate a degree of complexity or sophistication of a response to a query. For example, a machine learning model may be trained in a supervised or unsupervised manner to identify important features that could be used to correspond to different levels of complexity or sophistication of a response. For example, in a training set, one or more variables like response length, average number of letters per word, etc. may be used to compute a metric for complexity, and new features then may be identified by the model that may correspond to complexity or sophistication of a response to a query. In another, a generalized model may be fed a response of a subject, and the model estimates the IQ of the subject who provided the response.

In an example, an alert criterion may be defined that specifies, when to raise an alert to a service provider, based on the scores of one or more scores of metrics of responses. For example, the alert criterion may include a threshold, where an alert is to be generated when a metric or score exceeds a threshold, corresponding to the no or negligible neurotoxicity level. The workflow of generating an alert can include transmitting an email, text message, or an online message to the server or a combination of therefore, which can include an identification of a subject, one or more responses, and corresponding scores of one or more metrics that were used to determine that the neurotoxicity level of a subject is above the threshold of the negligible neurotoxicity level.

In an example, computing one or more scores of one or more metrics may be computed automatically on the user device of a subject, once the subject completes providing the responses to one or more queries on the user device. In an example, the responses to one or more queries may be transmitted, by the user device of a provider, to a service of a provider running in the cloud, and one or more scores of one or more metrics may be computed in the cloud. In an example, a subject may be scheduled to appear in person at the practice of a healthcare provider and provide the responses to one or more queries using the computer system of the provider, and one or more scores of one or more metrics may be computed on premise on the computer system of the provider. In an example, the responses to one or more queries may be transmitted, by the computer system of the provider, to a service of a provider running in the cloud, and one or more scores of one or more metrics may be computed in the cloud.

Once the neurotoxicity estimator determines that the neurotoxicity level of a subject is above the threshold of the negligible neurotoxicity level, AI model may use a rule-based expert system to prepare a comprehensive management plan for the subject (when the assessment was done at the practice of the provider) including but not limited to: (1) preparing an order of one or more lab investigations; (2) changing the treatment plan or suggesting discontinuing it, if need be; (3) presenting them to the physician for a review and approval; (4) and sending the lab orders to the designated labs and the updated treatment plan to the designated pharmacies once approved by the physician. In the case, when the assessment was undertaken remotely on the user device, the management plan may include scheduling an appointment of the subject with the provider, and transmitting analyses of assessments to the provider, and confirming to the subject the time of the appointment when approved by the assistant of the provider. The scores of one or more metrics, analyses performed on the scores of one or more metrics, and the inference about the neurotoxicity level of a subject may be stored in an EMR system housed on an on-premises server or a cloud server of the provider.

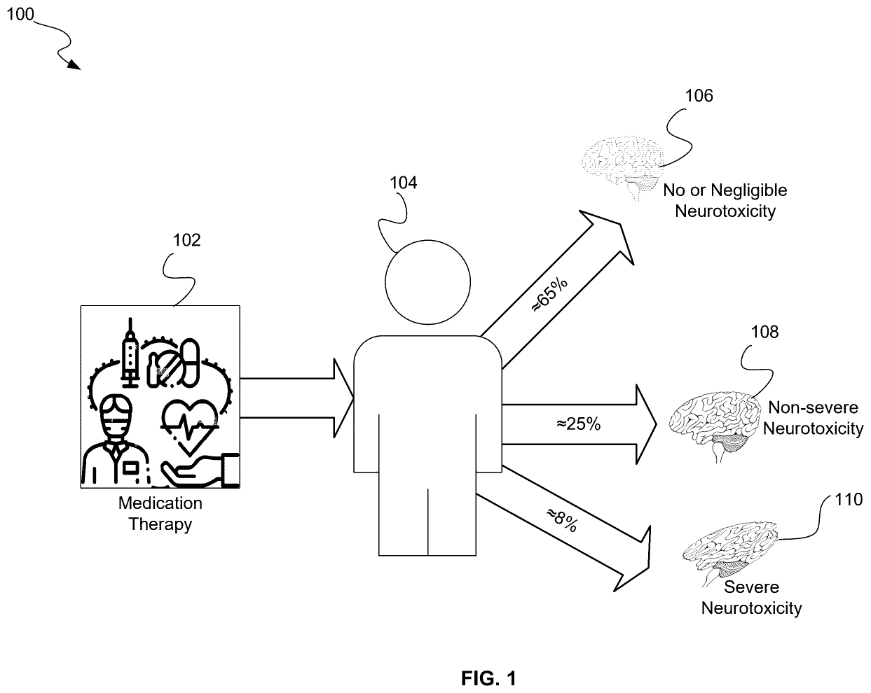

FIG. 1 shows a distribution of the subjects, for an example case study 100 developing neurotoxicity as a side effect of different medication therapies received by the subjects. The neurotoxic therapies may broadly fall into two categories: chemotherapies and antibiotics. Numerous other treatments may also cause nerve injuries, and each should be evaluated before administration. For example, CAR T-Cell therapy is a therapy that may be administered to subjects with different types of liquid tumors, such as lymphoma, leukemia, and multiple myeloma. One or more medication therapies in a set of medication therapy 102 may be administered to one or more subjects 104. In this example study 100, approximately 25% of the subjects experienced neurotoxicity levels corresponding to the ones in neurotoxicity 108, and 8% of the subjects who receive the treatment experienced severe neurotoxicity levels corresponding to the ones in severe neurotoxicity 110. While the remaining of 65% subjects may have no or negligible levels of neurotoxicity levels corresponding to the ones in no neurotoxicity 106.

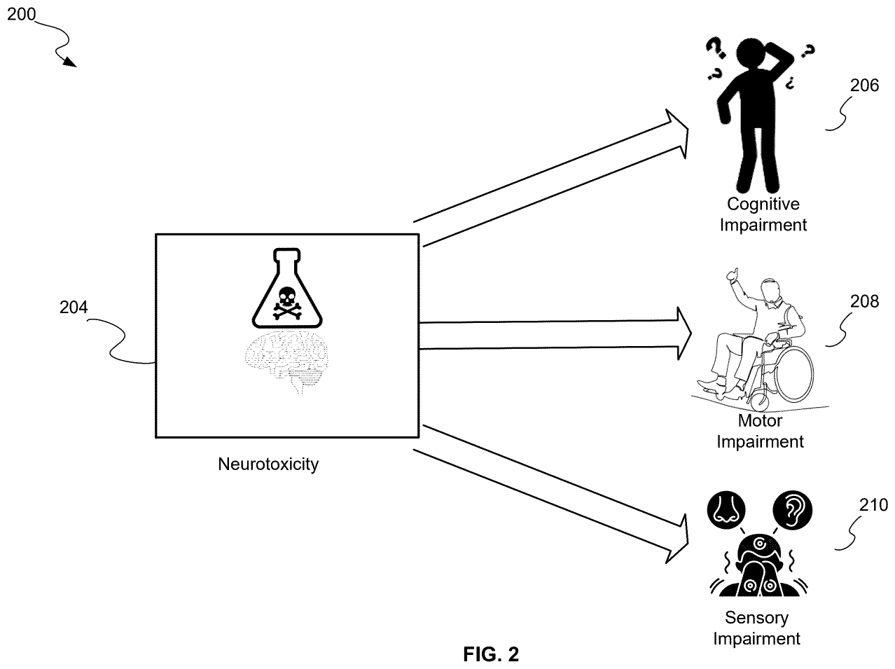

FIG. 2 shows one or more examples of different kinds of impairments experienced by the subjects due to neurotoxicity. These impairments may occur due to sustained level of neurotoxicity in subjects. Neurotoxicity may result in damaging brain or the peripheral nervous system of a subject, and the sustained neurotoxicity results in death of nerve cells, resulting in an irreversible damage to the brain. In this example 200, subjects having neurotoxicity levels corresponding to the ones in neurotoxicity 204 may experience cognitive impairment 206, motor impairment 208, or sensory impairment 210, etc. Consequently, subjects may suffer from mental illness such as anxiety, confusion, depression to name a few.

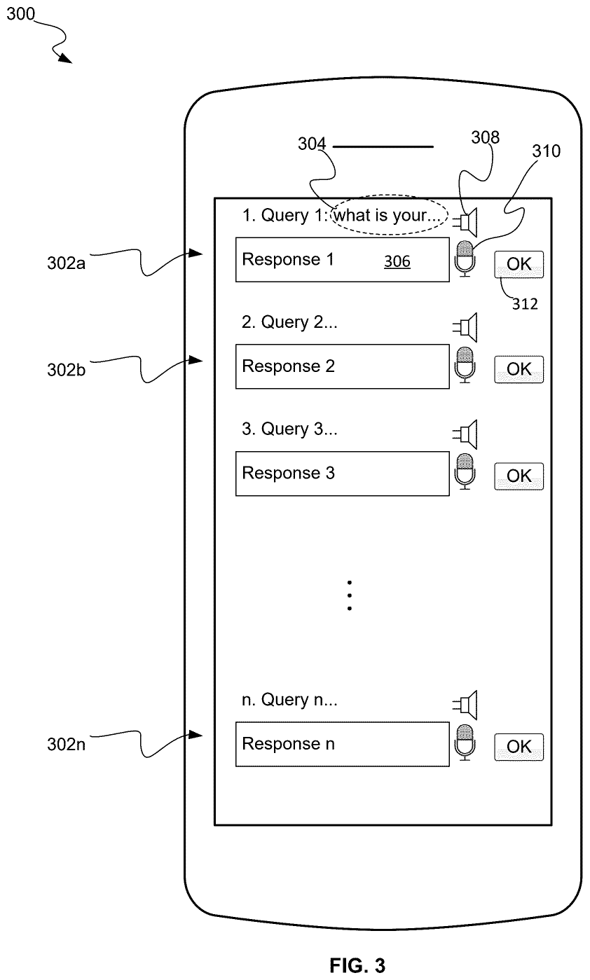

FIG. 3 shows an exemplary interface 300 of a neurotoxicity detector on a user device for performing neurotoxicity assessments for a subject by asking a plurality of questions and recording the respective responses. Various embodiments relate to new techniques, methods and systems for estimating neurotoxicity. Specifically, a computing device, such as a mobile device, desktop, or laptop may provide an exemplary interface 300 that is configured to present multiple queries to a user and to receive corresponding responses from the user. Interface 300 may present the subject with a set of queries 302a, 302b, . . . ,302n. All the queries may be shown to the subject at once, or one by one in a random manner. The queries may be or may include an open-ended question configured to receive a text based natural-language response. Thus, interface 300 may include, for each query, a natural language text of a question 304 and/or a corresponding text box 306 configured to receive the response in a text based natural language.

Additionally, or alternatively, the interface 300 may include different modes of input, for each query, such as the stored audio of a recitation of a query e.g., “Query 1” 302a that may be listened by the subject by pressing a component 308, and the subject can give a corresponding response e.g., “Response 1” by pressing component 310 and then the subject can speak into the microphone of the user device for recording the response. Alternatively, the response can also be typed in the provided input textbox corresponding to each query. After completing the response, the subject may indicate the completion of response by, for example, clicking or touching the “OK” button 312. When a response includes an audio signal, the audio signal may itself be analyzed directly or indirectly by transcribing into text that may be analyzed in accordance with the response-analysis technique disclosed herein. A subject can keep providing responses e.g., “Response 2”, . . . , “Response n” corresponding to the queries e.g., 302b, . . . , 302n, respectively by choosing text and voice-based input options by clicking on corresponding components in the interface 300.

Timings of subject sessions with the interface may be scheduled, for example, once per month, or twice per month, or midway between subsequent doses of the medication therapy, or may be recommended by a health practitioner. The contents of the set of queries may depend on several factors including, for example, knowledge, exposure level, education, experience, age, language, location etc. of the subject. Different sets of queries may be required to assess the responses of the specific subjects.

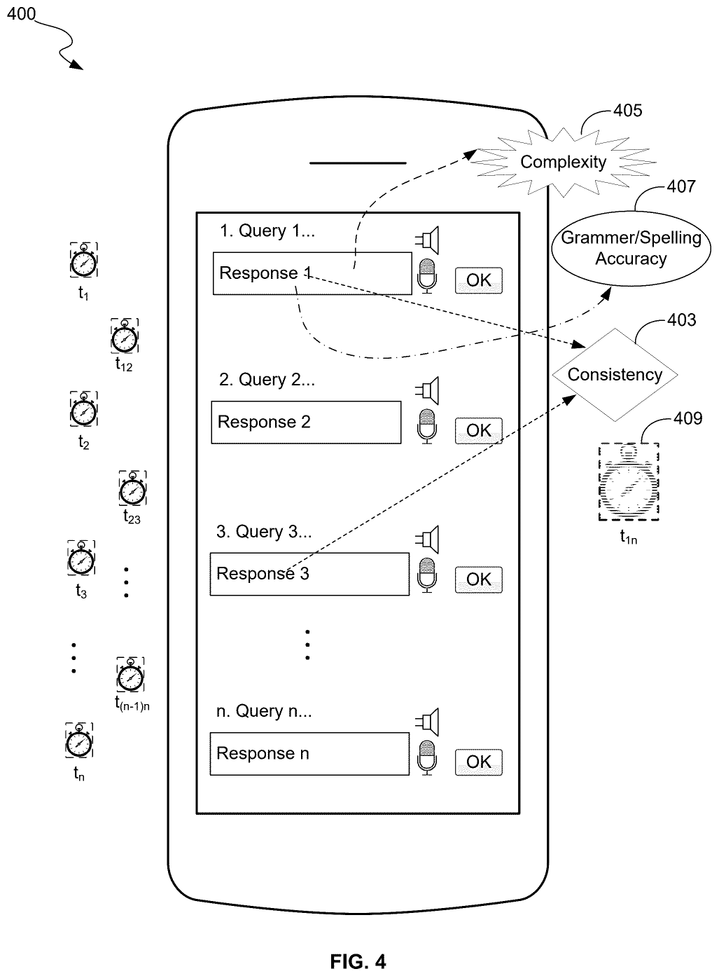

FIG. 4 presents exemplary assessments 400 performed on the one or more responses, received from the interface 300 of the user device of the subject. These assessments may be performed by response-analysis techniques that may include using one or more artificial intelligence (AI) techniques to assess different aspects of the responses provided by the subject. The assessments may include determining consistency 403 i.e., an extent to which responses provided by the subject in a given session with the interface are consistent with each other, complexity 405 or sophistication of responses provided by the subject in a given session with the interface 300, accuracy of grammar/spelling 407 i.e., a degree to which responses provided by the subject in a given session with the interface accord with grammatical rules and/or proper spelling, time durations t₁, t₂, . . . , tₙ taken by the subject to provide the corresponding responses to each of the n queries, an amount of time that a user spent providing responses during a single session i.e., t₁ₙ (=Σtᵢ) 409, pause times i.e., the time subject paused while providing corresponding responses to two consecutive queries e.g., t₁₂, t₂₃, . . . , t₍ₙ₋₁₎ₙ, where t₁₂ denotes a pause time taken by the subject between response 1 and response 2, and cumulative amount of time Σtᵢ₍ᵢ₊₁₎ that a user paused while providing responses to all n queries during a given session.

To assess the complexity of a response the subject may be asked a question, for example, “Are you experiencing any changes in your movement ability?”. The subject may reply in one or more different ways: (1) “Yes”; (2) “Yes, I am not able to control my movements effectively”; and (3) “Yes, I've been experiencing clumsiness, and my hand keeps on shaking while using soldering iron”. The first of these responses is a simple reply, the second one is relatively complex, while the third one is the most complex of the exemplary three responses. Based on the complexity of a response, the first response may be assigned a low complexity score, the second response may be assigned a medium complexity score, while the third response may be assigned a high complexity score. High complexity scores may be expected in the initial stages of a medication therapy, while low scores may be expected in subsequent stages of the medication therapy due to deterioration in mental health.

In order to estimate the consistency of the responses, two or more questions having the same answer may be asked in different ways: (1) “Who is the current president of the USA?”; (2) “Who was the winner of last presidential elections of the USA?”; and (3) “What is name of the husband of the current first lady of the USA?”. The answers to all the three questions are the same. If the subject replies with the same answer to all the three questions, then the replies will be considered consistent, and a high consistency score may be awarded. If the replies are different from each other, then the consistency score may be significantly lower compared to the scenario when the answer was the same.

Additional or alternative assessments may characterize the degree to which each of one or more of the above-noted variables differs from corresponding variables associated with prior sessions of a same user. In some instances, each of two or more of these assessments, by a same artificial intelligence model or by separate assessment specific artificial intelligence models, are performed, to result in a performance metric for the particular assessment. A score may then be generated using one or more performance metrics.

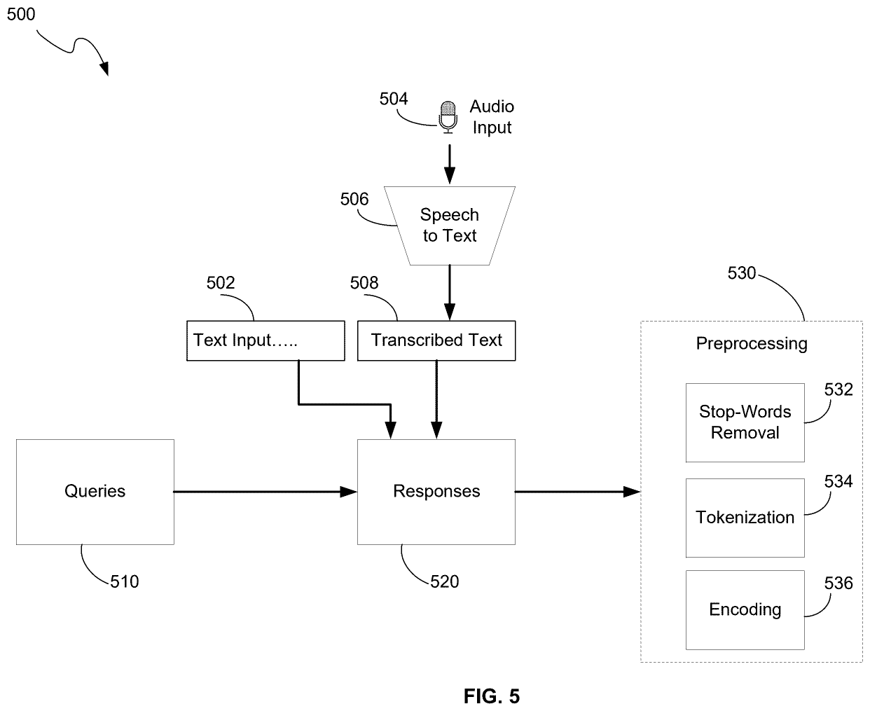

FIG. 5 illustrates an exemplary preprocessing pipeline 500 for the one or more responses 520 received from the user device of the subject. These responses 520 may be received in response to the one or more queries 510 in text form 502 or audio form 504 during a single session of a subject with the interface 300. One or more artificial intelligence (AI) techniques may be used to perform the preprocessing steps. For example, if one or more of the responses are in audio format, the speech to text conversion 506 may be performed to convert the audio input 504 to its corresponding transcribed text 508. Speech to text conversion may be performed by leveraging one or more machine-learning (ML) models, for example, recurrent neural networks (RNNs), long short-term memory (LSTM) networks, and transformer-based models, or by using services like Google® Cloud Speech-to-Text, Amazon® Transcribe, and Microsoft® Azure Speech to Text etc. for automatic speech to text conversion. The text input 502 or the transcribed text input 508 may be recorded as a response in a list of one or more responses 520 for the corresponding queries in a list of one or more queries 510. Further preprocessing 530 may be performed on one or more responses 520 that may involve (for example) stop-words removal 532 (e.g., a, an, the, is etc.) and/or tokenization 534 that include transforming various words or combination of words into corresponding tokens (e.g., using a library). The tokens may then be transformed into vectors using an encoding technique 536 (e.g., term frequency-inverse document frequency (TF-IDF) vectorizer, Word2vec, skip-gram, GloVe). The encoding technique 536 may be configured to perform the transformation based on the occurrence frequency of a given token (or related tokens) in a given response (or a combination of responses) and/or based on occurrence frequency of a given token (or related tokens) in an underlying set of responses that may be generated based on responses from many users, and from many assessments of the same users.

It will be appreciated that audio input need not be converted to text to be processed. For example, audio files may be processed to generate one or more statistics relating to sense tone, pauses, a degree to which various detected pauses reliably correlate with response time (versus being more variable), and/or pitch variability.

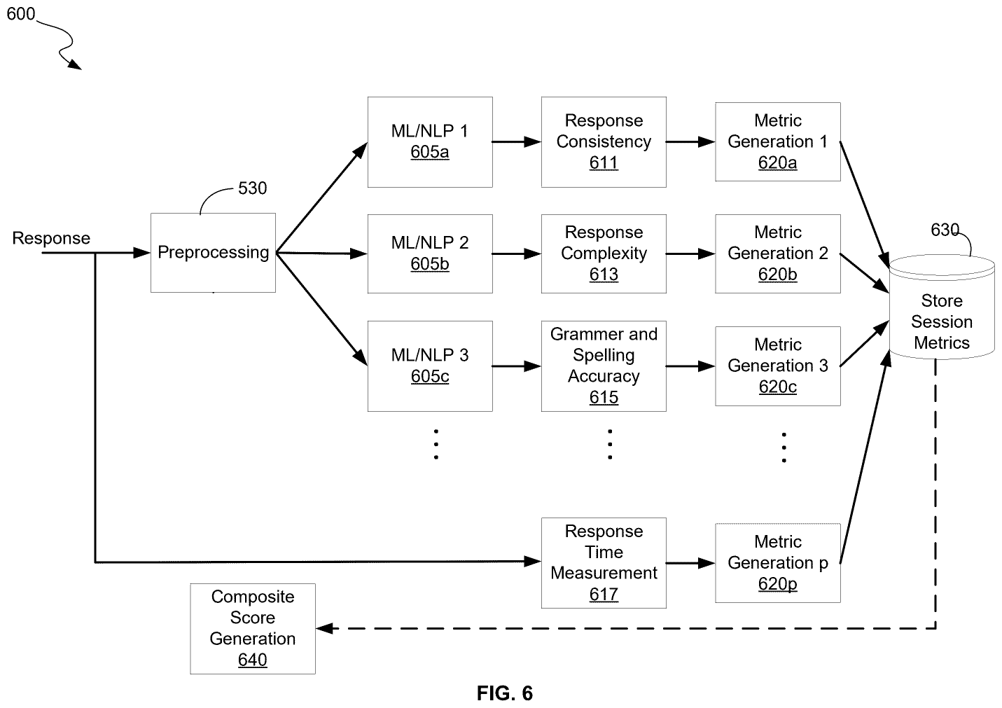

FIG. 6 illustrates an example method 600 of computing one or more scores associated with different metrics of the respective responses of the subject during a single session, received from the interface 300 of the user device of the subject in accordance with some embodiments of the present disclosure. Preprocessing 530 to the one or more responses 520 may result in multi-dimensional encoding vectors that can be fed to one or more ML/NLP techniques 605a, 605b, . . . , 605c that based on features may generate one or more assessment metrics for the responses 520 provided by the subject. The features may include, for example, determination of tokens that are common in a response set for a given subject but less common in the underlying set, so that such tokens may be interpreted as being relatively important for conveying meaning of the response set. Further, the underlying data set may be analyzed to identify token pairs or token combinations that are relatively frequent in a same response, response set, webpage, etc. (and/or within a given distance from each other), and token pairs and/or token combinations can then be evaluated in view of the underlying data-set analysis results to quantify (for example) a predicted response consistency, response complexity, etc.

The assessment may include metrics such as response consistency 611, response complexity 613, and grammar and spelling accuracy 615. The one or more ML/NLP models may include using one or more trained models, which may include a generative model, a neural network, a long-short term memory model, a transformer model, a moving-average model (e.g., an autoregressive integrated moving average (ARIMA) model), a model that uses self-attention, such as ChatGPT, etc. In instances where different trained machine learning models are used to generate different metrics: two, more or all of the different trained machine learning models may be of a same type of model and/or include a same architecture and/or two, more or all of the different trained machine learning models may be of different types of models and/or include different architectures. The (assessment) metrics may also include response time measurement 617 of time durations t₁, t₂, . . . , tₙ taken by the subject to answer each of the n queries, an amount of time tₘₙ that a user spent providing response n during m session, pause times t₁₂, t₂₃, . . . , t₍ₙ₋₁₎ₙ the subject paused while providing response to two consecutive queries, and cumulative amount of time Σtᵢ₍ᵢ₊₁₎ that a user paused while providing responses during a given session.

In some instances, a consistency between responses 611 (across responses provided to queries presented in a single session with the interface 300 or across responses from different sessions) may be estimated by comparison of tokens in responses. For example, tokens may be mapped to corresponding positions in a multi-dimensional space by leveraging an encoding technique (using e.g., TF-IDF vectorizer, Word2vec, skip-gram, GloVe etc.) based on a baseline dataset. The position can be represented as a vector in the multi-dimensional space, for example, a multi-dimensional Euclidean space. The consistency of responses may be estimated based on the location of the tokens across the space; a comparison of a first distribution of positions (e.g., corresponding to tokens from a particular response or particular session) relative to a second distribution of positions (e.g., corresponding to tokens from a different particular response or different particular session); calculating one or more statistics based on distances between representations of tokens (e.g., across responses in a single session or across responses from multiple sessions), etc.

In some instances, a generative model may be used to predict a response to a query based on a response from one or more other queries (e.g., from a same session or one or more previous sessions). For each of the predicted response and an actual response, stopwords may be removed and tokens may be generated. Each token can be projected to a position in a multi-dimensional space (using e.g., TF-IDF vectorizer, Word2vec, skip-gram, GloVe etc.). The position can be represented as a vector in the multi-dimensional space, for example, in a multi-dimensional Euclidean space. The consistency of responses 611 may be estimated based on a degree to which a distribution of tokens from one or more actual responses differs from a distribution of tokens from one or more corresponding predicted responses. A consistency of responses can alternatively or additionally be estimated based on a distance between projections of tokens in the actual responses relative to projections of tokens in the predicted responses.

In some instances, a machine learning model or rule-based model may be used to estimate an absolute or relative response complexity 613 (or sophistication) of one or more responses. For example, a model may be trained and configured to associate each of one or more tokens or token combinations with a complexity level. To illustrate, the model may be trained to associate tokens from a first subset of training data (e.g., scientific manuscripts) with higher complexity metrics than tokens from a second subset of training data (e.g., social-media posts) having lower complexity metrics. As another example, the model may be trained in an unsupervised fashion to learn how various content features correspond with complexity. As yet another example, one or more rules may be defined to calculate a complexity or sophistication of one or more responses based on: a distribution of syllables per word, a distribution of words per sentence, how frequently various words or phrases are used in a baseline dataset, or the type(s) of punctuation that is used. In some cases, response complexity 613 may be measured by supposing the vectors in a response as a set of vectors, and estimating the mean, variance, or spatial spread of the set of vectors, or by performing principal component analysis (PCA) and/or independent component analysis (ICA) to identify critical directions in a subspace of the multiple dimensional space.

In some instances, a machine-learning or rule-based model can estimate a degree of response complexity 613. For example, a machine learning model may be trained (e.g., in a supervised or unsupervised manner) to identify features that correspond to different levels of complexity. For example, in a training set, one or more variables (e.g., response length, average number of letters per word, etc.) may be used as a metric for complexity, and new features may then be identified by the model that correspond to complexity or sophistication. As another example, a generalized model may be fed a response and requested to output an IQ or grade level of a person who provided the response.

In some instances, each of two or more of these assessments (e.g., by a same artificial intelligence model or by separate assessment-specific artificial intelligence models) are performed, so as to result in metrics for the particular assessment. A score may then be generated using the metrics. For example, score generation 620a, 620b, . . . , 620p are p scores generated corresponding to the assessments performed above. The scores may each be a scalar quantity or may be a vector having dimensions greater than one. The scores may be stored, for each session with the interface 300, in a storage 630 (e.g., a non-transitory storage) for further consultation or comparison or computing an aggregate score by aggregator 640.

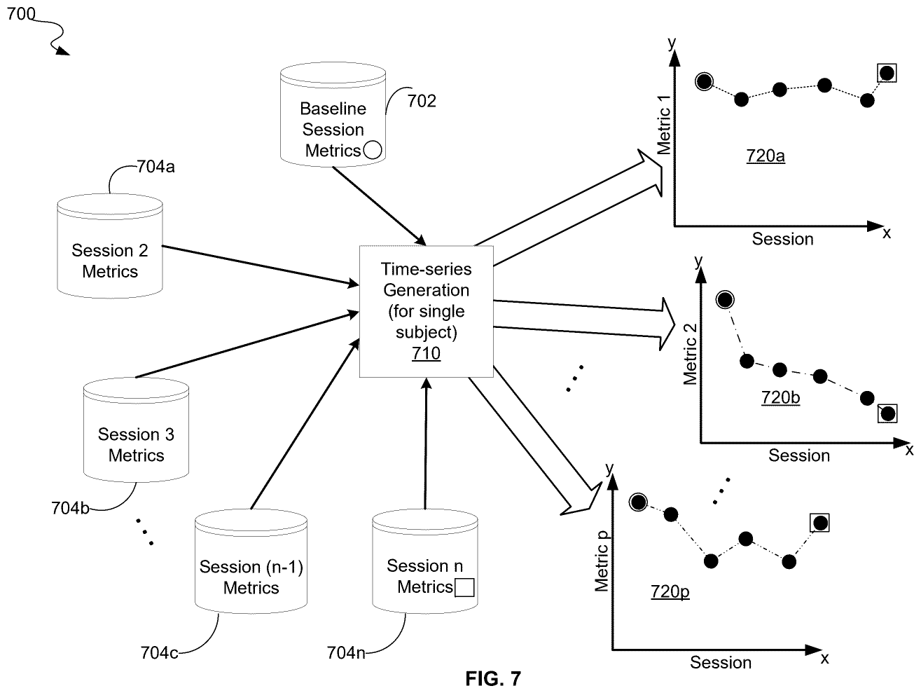

FIG. 7 is an example illustration of determining assessments for the respective responses in inter-session analysis based on the one or more scores associated with different metrics. In this example 700, baseline session scores 702 of a metric, that may represent an average of a score across multiple sessions of one or more subjects, may be used for a subject even before starting a medication therapy. In some cases, a subject may have started receiving medication therapy before taking a session of assessments using the interface 300 on a user device. In such cases, a first session during the course of the medication therapy may be taken as the baseline session for the subject. The scores of metrics of one or more subsequent sessions, 704a, 704b, . . . ,704n, of the subject may be generated and stored in a storage corresponding to the session identifier. The scores of a current session 704n may be compared with the scores of one or more previous sessions 704a, 704b, . . . ,704(n−1). The patterns of one or more scores, corresponding to one or more metrics (consistency, complexity, grammar/spelling, response time) may be generated by using AI models to conduct inter-session analysis 710 for a particular subject. The patterns and trends in one or more scores may be determined, for example, FIG. 7 shows graphs 720a, 720b, . . . , 720p illustrating patterns, where each graph of the one or more graph shows the patterns or trends for a single score. For example, plot 720a shows the variation trends of score 1 tiacross one or more sessions of a particular subject. Similar trend plots may be generated for score 2 to score p across one or more sessions of a particular subject. The plot 720b and plot 720p show the variation trends of score 1 and score p respectively across one or more sessions of a particular subject.

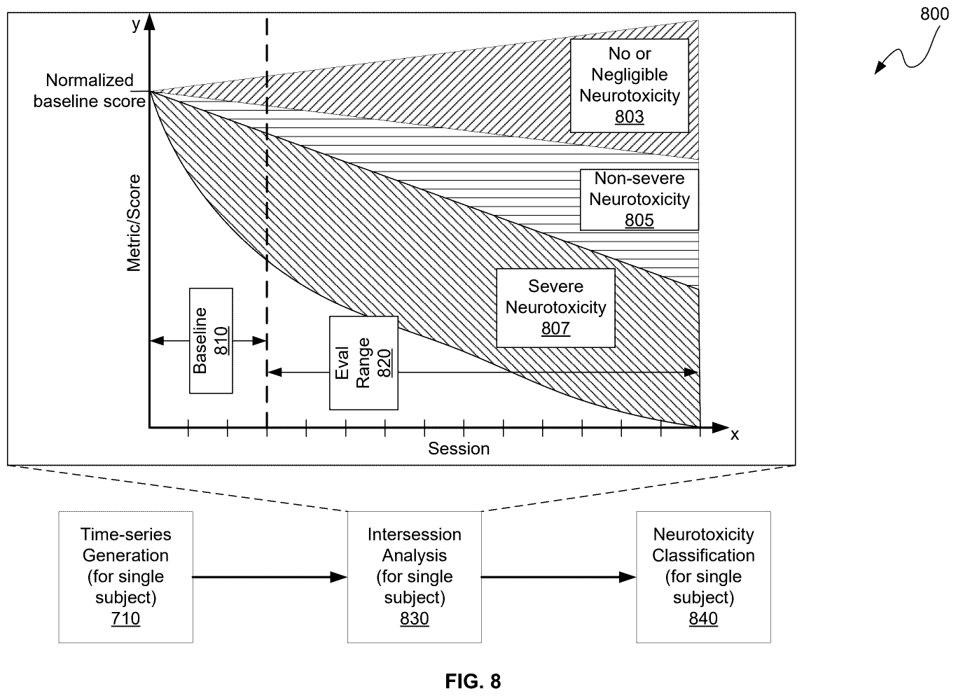

FIG. 8 shows an example graph 800 illustrating how a computed score of a metric across different sessions can be compared with a baseline score of the metric to estimate a level of neurotoxicity in a subject. The score on the y-axis (called score axis) may be any one of the one or more scores corresponding to one of the one or more attributes (consistency, complexity, grammar/spelling, response time). All the scores may be recorded for one or more sessions for a particular subject using the interface 300 on a user device. The scores of first few sessions of the one or more sessions may demonstrate a transient response in the transient range 810, whereas the scores of the remaining sessions of the one or more sessions, after transient range 810, may demonstrate a steady state response in a steady state range 820. Region 803 in the graph 800 may correspond to a range of score values having no or negligible neurotoxicity. Region 805 in the graph 800 may correspond to a to a range of score values having non-severe neurotoxicity. In comparison, region 807 may correspond to a range of score values having severe neurotoxicity. Once the transient response of a score of a metric is finished, and the steady state scores of a particular subject lie in one of the regions 803, 804, or 807, and remain in the same region for the remaining sessions of the one or more sessions until the start of a current session, then the subject may be classified to have a label of the corresponding region—negligible neurotoxicity or non-severe neurotoxicity or severe neurotoxicity—showing the neurotoxicity level of the subject. In one example, the baseline score of a metric may be normalized to a numeric value, and the scores of subsequent sessions of the one or more sessions may be normalized relative to the baseline score.

In the beginning of few sessions of a particular subject, if a score lies in a first region associated with any one of the three regions 803, 805, or 807, transits to a second region associated with a different region from the first region, and remains in the second region for the remaining sessions of one or more sessions, then the particular subject may be classified to have the neurotoxicity level corresponding to the label of the second region. For example, if the steady state score of a metric of a particular subject remains in the region 803 (e.g., no or negligible neurotoxicity) until the 5^{th} session, and then the value of the score starts changing at the beginning of the 6^{th} session such that the score moves to the region 807 (e.g., severe neurotoxicity), and then it remains in the same region during the 6^{th}, 7^{th} and 8^{th} sessions, then this can be safely inferred that the particular subject has the severe neurotoxicity level.

In an example, where a steady state score of a particular subject starts in a first region of the three regions 803, 805, or 807 and remains in that region for one or more session, and then the steady state score moves to a second region, different from the first region, of the three regions 803, 805, or 807, and remains in the second region for one or more sessions, and finally the steady state score again transits to the first region from the second region, and remains in that for one or more remain sessions: it may be an outlier situation. The reason this scenario may represent an outlier situation could be because of one or more factors: ambient environment around a particular subject during a session, signal interference from the sound of the other people nearby a particular subject, loss of internet connection, or short lived psychological conditions that may not be linked to the medication therapy but may adversely influence the response of a particular subject. Outliers can be detected using statistical techniques such as z-score, modified z-score, or Tukey's method to identify outliers based on the standard deviation of the score from the mean or median of a sequence of scores. Distance-based methods like the k-nearest neighbors method may also be used to detect outliers by measuring the distance of each point, corresponding to a score value of a metric, in a multidimensional space to its k-nearest neighbors. If the distance is above a threshold, the point is treated as an outlier. Outliers may also be detected by first clustering scores corresponding attributes in a training dataset and then outliers are the scores that either do not belong to a cluster or belong to only a small number of clusters. Supervised learning models such as support vector machines (SVMs) and random forests may also be configured during training to detect outliers.

An alert may be generated if a steady state score of a particular subject remains in the region 805 corresponding to non-severe neurotoxicity or region 807 corresponding to severe neurotoxicity for two consecutive sessions in the steady state region 820. The alert may be sent to a user device of a particular subject and may also include recommending to the subject to schedule a consulting session with a neurologist to discuss and for confirmation of the level of neurotoxicity predicted the regions-based method 800. During the consultation session with a neurologist, a particular subject can also discuss one or more management options including changing the treatment plan to slow down the progress of neurotoxicity, or even bring it back to the negligible neurotoxicity level if such a possible option exists.

In another example, a majority of steady state scores for one or more metrics may indicate no or negligible neurotoxicity, but at least one score may show severe neurotoxicity. In a conservative and aggressive regime, an alert may still be generated, as it is safe for a subject to visit a neurologist and then it is confirmed that the subject has no or negligible neurotoxicity. This situation is better than the one where an alarm was not generated, and a subject was actually suffering from severe neurotoxicity.

An aggregate score for a particular subject may be also generated from a set of one or more scores for a single session. The aggregate score may, for example, be the mean, weighted mean, median, mode, or geometric mean of the set of one or more scores for a single session. A sequence of aggregate scores of a particular subject across one or more sessions may be generated and the graph method 800 be used to infer the level of neurotoxicity. An average or moving average of a sequence of aggregate scores may also be used to infer the neurotoxicity level of a particular subject.

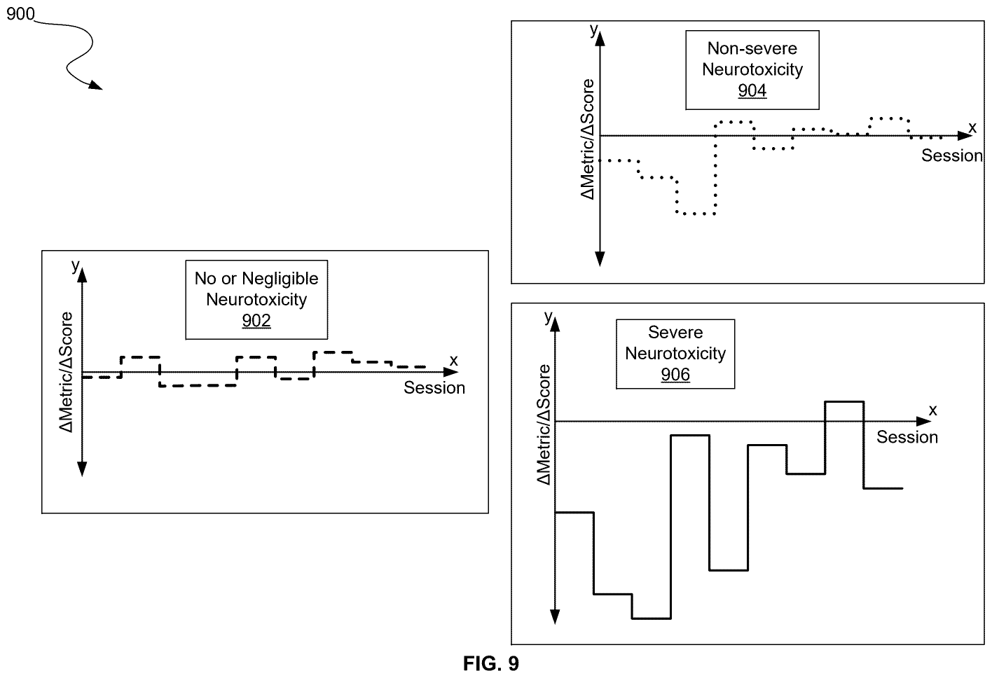

FIG. 9 shows one or more exemplary graphs 900 to illustrate how a rate of change of the computed score of the metric across different sessions can be used to estimate the level of neurotoxicity in the subject. In this case, the rate of change or a derivative of a score (or an aggregate score) across multiple sessions may be used to predict a neurotoxicity level of a particular subject. In example plot 902, the rate of change of a score across multiple sessions remains low and its values oscillate close to x-axis. As a result, the absolute value of the ratio between total positive area and total negative area

$$\vert \frac{total positive area}{total negative area} \vert$$

may be close to 1, where total positive area and total negative area are the positive and negative areas between the Δscore graph and the horizontal axis. Consequently, this subject might be classified as having no or negligible neurotoxicity 902.

In example plot 904, the rate of change of a score across multiple sessions remains large in the beginning sessions and then gets smaller in the later sessions. In this graph 904, the derivate values of a score in approximately half of the sessions are negative and are below the x-axis, and in approximately half of the sessions the derivate values of the score also become slightly positive and goes slightly above the x-axis. As a result, the absolute value of the ratio between the total positive area and total negative area may be close to 0.5. In some instances, a condition may be configured such that the initial decline (e.g., quite consistent decline) is sufficient to result in a classification of severe neurotoxicity. For example, empirical data may suggest that the initial decline is sufficient to result in a classification of predicted severe neurotoxicity or irreparable neurotoxicity. In some other instances, a condition may be configured such that the initial negative derivative values are insufficient to indicate that any irreparable or physiologically noticeable neurotoxicity is to be assigned for a classification. Therefore, if the changes in scores thereafter resort towards zero or positive, a classification of no or negligible neurotoxicity may be assigned. In yet other instances, a condition may be configured such that the cumulative derivative values indicate that a predicted non-severe neurotoxicity is to be assigned for a classification.

In plot 906, the rate of change of a score remains consistently high on the negative side across all sessions. In this graph, the derivate values of a score for most of the sessions are negative and are well below the x-axis, and only for a small number of sessions they may be slightly positive above the x-axis. As a result, the absolute value of the ratio between total positive area and total negative area

$$\vert \frac{total positive area}{total negative area} \vert$$

may be close to 0. Consequently, this subject might be classified as having severe toxicity.

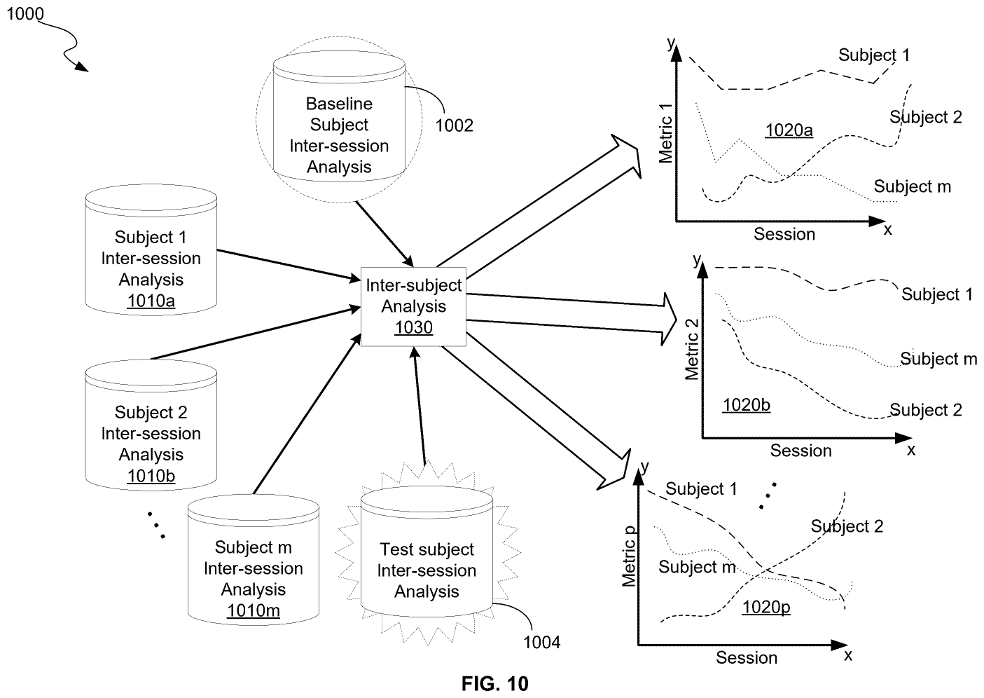

FIG. 10 shows estimation of neurotoxicity level in a test subject 1004 by comparing the scores of test subjects 1004 with the scores of m other subjects 1010a, 1010b, . . . , 1010m who may also have developed neurotoxicity and the scores of a baseline subject 1002. The inter-session analysis of the baseline subject 1002 may be generated using method 700 over a period of time. The baseline subject may have either not received any medication therapy or received a placebo medication therapy. In some cases, the baseline subject 1002 may not be even a real person, rather the baseline subject 1002 can be a hypothetical person generated by leveraging ML clustering techniques. Inter-subject analysis 1030 may be performed by comparing the inter-session scores of test subjects 1004 with the inter-session scores of the baseline subject 1002 and inter-session scores of one or more subjects: subject 1 1010a to subject m 1010m. The trends and patterns of various scores, corresponding to various attributes, may be generated by leveraging one or more statistical techniques to produce inter-subject analysis 1030 for the test subject 1004. The trends and patterns of different scores may be shown in the form of graphs 1020a, 1020b, . . . , 1020p, where each line in a graph shows the trend of a score for a subject. The patterns of score 1, score 2, . . . , score p of test subject 1004 may show a possible decline in the quality of the attributes of test subject 1004 when compared with other m subjects, 1010a, 1010b, . . . , 1010m who may have developed neurotoxicity and baseline subject 1002 who did not receive a medication therapy. A distance measure may be used to determine the closeness of test subject 1004 to baseline subject 1002 or one or more m subjects; as a result, the neurotoxicity level of test subject 1004 may be predicted.

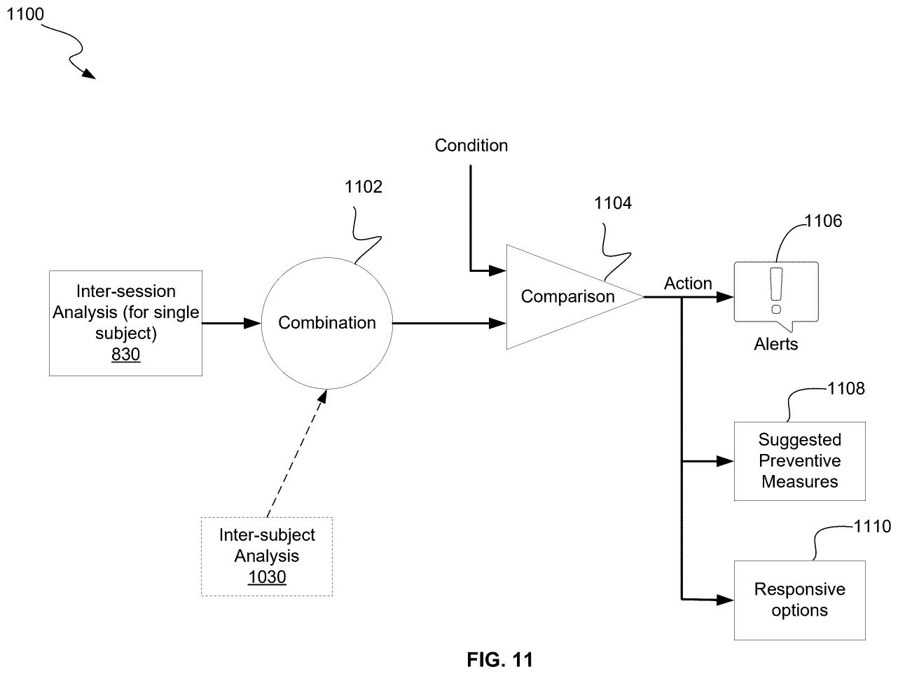

FIG. 11 shows generating one or more actions when neurotoxicity in a subject is detected. The inter-session analysis 710 of a subject may be further combined at 1102 with inter-subject analysis 1030 and then the presence of a condition is detected by a comparator 1104. The condition may include ranges of scores or predefined thresholds that correspond to negligible neurotoxicity 803, non-severe neurotoxicity 805, or severe neurotoxicity 807. Based on the comparison results, one or more actions may be triggered that may include, for example, an alert message 1106, suggesting one or more preventive measures 1108, and/or trigger one or more response actions 1110. An alert criterion may be defined that specifies, based on one or scores of metrics, whether the alert 1106 is to be generated. For example, the alert criterion may include a threshold, where the alert 1106 may be generated when a metric or score exceeds a threshold. The alert may be generated when a subject's scores enter into a cluster associated with increased neurotoxicity. The alert may be generated when a subject's score changes from being associated with one cluster to being associated with another cluster. In some instances, a level of toxicity may be estimated for each cluster, and an alert may be generated when a subject's score transitions across associations with clusters that are estimated to have a difference in toxicity levels that exceed a predefined (e.g., received or learned) threshold. Generating the alert 1106 can include, for example, sending an email, SMS message, or online message, any or all of which may include an identifier of a subject, include one or more responses (and/or queries), one or more metrics, and/or one or more scores. In some instances, each of one or more metrics and/or one or more scores are output (e.g., transmitted to or presented at a device of the subject and/or a corresponding healthcare provider). Such an output may occur automatically upon completion of processing of the responses, at a scheduled time, or upon request from a user or a healthcare provider.

The metric(s) and/or scores may be used to automatically trigger one or more response options 1110, such as preparation of an order of one or more lab tests, a changed prescription, a scheduling of an appointment of a subject with a healthcare provider, etc. In some cases, the order or prescription may be automatically sent to the designated labs or pharmacies, and also the appointment of the subject with the healthcare provider is scheduled and confirmed. In some cases, the order, prescription, or appointment request may be prepared by an AI-based expert system; and then a request is sent to a healthcare provider to approve or edit response actions. Once the healthcare provider approves the response actions, only then the order or prescription may be sent to the designated labs or pharmacies respectively. Similarly, an appointment with a neurologist can only be confirmed once the neurologist approves it. Consequently, various examples may use statistical techniques or artificial intelligence to quickly and efficiently detect the level of neurotoxicity in a subject and to initiate response actions to stop or reverse the effects of neurotoxicity if possible.

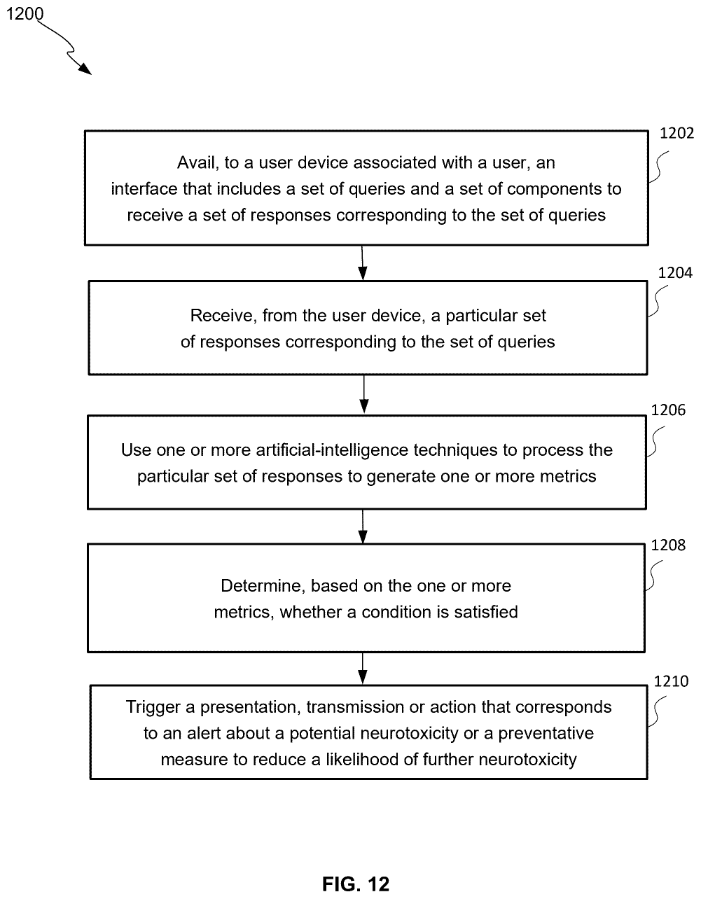

FIG. 12 illustrates an example flowchart of method 1200 for determining the level of neurotoxicity in a subject by leveraging interface 300 on a user device that is associated with the subject. The blocks in the flow chart of method 1200 are illustrated in a specific order, while the order can be modified, for example, some blocks may be performed before other, and some blocks may be performed simultaneously. The blocks can be performed by hardware or software or a combination thereof. The process 1200 may include an interface that includes a set of queries and a set of components to receive a set of responses corresponding to the set of queries, at block 1202. The set of queries may be presented in a text form or in a form of one or more audio recordings including a recitation of a query. The set of responses may be received either in a text form or in an audio form. At block 1204, the set of responses from the user device may be received at a server which may be on the user device or may be a remote server in a cloud. The server performs steps of a preprocessing pipeline and does feature engineering to generate important features form the set of responses. One or more artificial intelligence techniques may be used to process features corresponding to a particular set of responses to generate one or more metrics, at block 1206.

At block 1208, the one or more metrics may be analyzed to determine whether a condition is satisfied. At block 1210, if the condition at block 1208 is satisfied, an action may be triggered which may include a presentation, transmission or action that corresponds to an alert about a potential neurotoxicity or one or more preventative measures that may likely reduce the level of neurotoxicity in a subject, and if possible, bring it to a negligible neurotoxicity level.

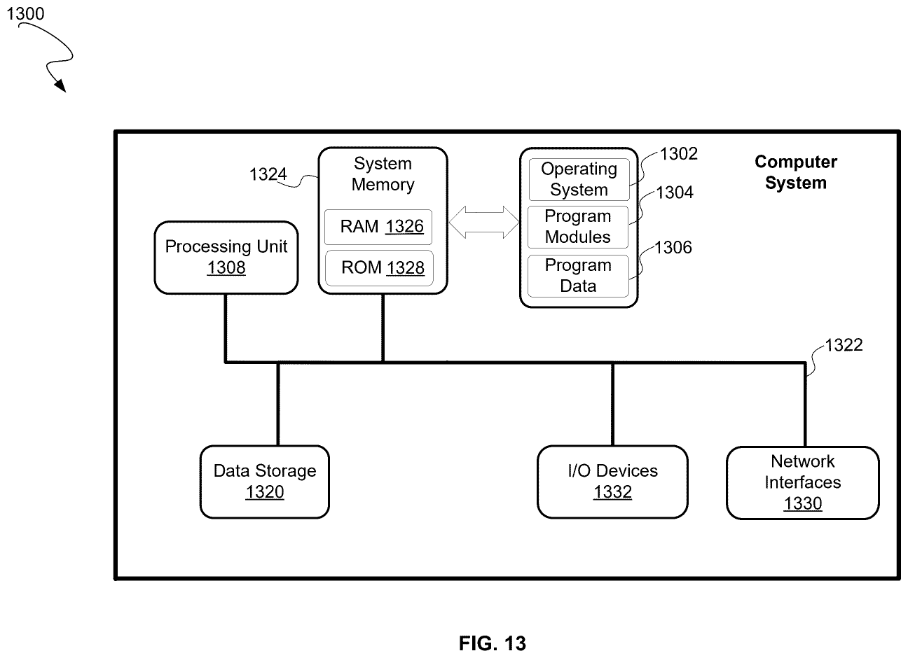

FIG. 13 is an example illustration of a computer system 1300 in which various embodiments of the present disclosure may be implemented. For example, the techniques described above such as availing a user interface, presenting a set of queries, receiving a set of responses, preprocessing of the responses, feature generation, score generation, score analysis, and triggering actions etc. can be implemented in computer-executable instructions (e.g., organized in program modules 1304). The program modules 1304 can include the routines, programs, objects, components, and data structures that perform the tasks and implement the data types for implementing the techniques described above. The functionality described herein can be performed, at least in part, by one or more hardware logic components.

To provide additional context for various aspects, FIG. 13 and the following description are intended to provide a brief, general description of the suitable computer system 1300 in which the various aspects can be implemented. While the description is in the general context of computer-executable instructions that can run on one or more computers, those skilled in the art will recognize that a novel implementation also can be realized in combination with other program modules and/or as a combination of hardware and software. The computer system 1300 for implementing various aspects includes a processing unit 1308 having one or more processors (also referred to as microprocessors), a computer-readable storage medium (where the medium is any physical device or material on which data can be electronically and/or optically stored and retrieved) such as a data storage unit 1320 (computer readable storage medium/media also include magnetic disks, optical disks, solid state drives, external memory systems, and flash memory drives), and a system bus 1322. The system bus 1322 may provide an interface for system components including, but not limited to, system memory 1324, to processing unit 1308. Such a system bus 1322 can be of any of several types of bus structure that can further interconnect to memory bus (with or without controller), and a peripheral bus (e.g., PCI, PCIe, AGP, LPC, etc.), using any of a variety of commercially available bus architectures.

FIG. 13 shows an example configuration of a typical computer that may be other commercially available microprocessors such as single-processor, multi-processor, single-core units, and multi-core units of processing and/or storage circuits. Moreover, those skilled in the art will appreciate that the novel system and methods can be practiced with other computer system configurations, including minicomputers, mainframe computers, as well as personal computers (e.g., desktop, laptop, tablet PC, etc.), hand-held computing devices, microprocessor-based or programmable consumer electronics, and the like, each of which can be cooperatively coupled to one or more associated devices.

In some aspects, the computer system 1300 can be one of several computers employed in a datacenter and/or computing resources (hardware and/or software) in support of cloud computing services for portable and/or mobile computing systems such as wireless communications devices, cellular telephones, and other mobile-capable devices. Cloud computing services, include, but are not limited to, infrastructure as a service, platform as a service, software as a service, storage as a service, desktop as a service, data as a service, security as a service and APIs (application program interlaces) as a service, for example. In some instances, system memory 1324 can include computer-readable storage (physical storage) medium such as a volatile memory (e.g. random-access memory (RAM) 1326) and a non-volatile memory (e.g., (ROM) 1328). A basic Input/output system (BIOS) can be stored in the non-volatile memory and includes the basic routines that facilitate the communication of data and signals between components within the computer system 1300, such as during startup. The volatile memory also includes a high-speed RAM such as static RAM for caching data.

By way of example, and not limitation, system memory 1324 also may also include program modules 1304, which may include client applications, Web browsers, mid-tier applications, relational database management systems (RDBMS), etc., program data 1306, and an operating system 1302. By way of example, operating system 1302 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome OS, and the like) and/or mobile operating systems such as iOS, Windows® Phone, Android OS, BlackBerry® OS, and Palm® OS operating systems. All or portions of operating system 1302, program modules 1304, and/or program data 1306 can also be cached in memory such as the volatile memory and/or non-volatile memory, for example (RAM 1326 or ROM 1328). It is to be appreciated that the disclosed architecture can be implemented with various commercially available operating systems or combinations of operating systems (e.g., virtual machines).

In some other examples, the computer system 1300 may have additional features or functionality. For example, the computer system 1300 may also include additional data storage devices (removable and/or non-removable) such as, for example, magnetic disks, optical disks, or tape. Computer-readable media may include, at least, two types of computer-readable media, namely computer storage media and communication media. Computer storage media may include volatile and non-volatile, removable, and non-removable media implemented in any method or technology for storage of information, such as computer readable instructions, data structures, program modules, or other data.

The system memory 1324, and data storage 1320 including removable storage, and non-removable storage are all examples of computer storage media. Apart from RAM 1326 and ROM 1328, computer storage media includes, but is not limited to, EEPROM, flash memory or other memory technology, CD-ROM, digital versatile disks (DVD), or other optical storage, magnetic cassettes, magnetic tape, magnetic disk storage or other magnetic storage devices, or any other non-transmission medium that can be used to store the targeted information and which can be accessed by computer system 1300. Moreover, the computer readable media may include computer-executable instructions that, when executed by the processing unit 1308, perform various functions and/or operations described herein. In contrast, communication media may embody computer readable instructions, data structures, program modules, or other data in a modulated data signal, such as a carrier wave, or other transmission mechanism.

The computer system 1300 may also include one or more input/output I/O devices 1332. The one or more input devices of the one or more I/O devices 1332 may be, for example, keyboard, mouse, pen, voice input device, touch input device, etc. The one or more output devices of the one or more I/O devices 1332 may be, for example, display, speakers, printers, etc. may also be included. These devices are well known in the art and are not discussed at length here. The computing device 1300 may also include one or more network interfaces 1330 to establish communication that may allow computer system 1300 to communicate with other system or devices, such as over a network. These networks may include wired networks as well as wireless networks. Here, the computer system 1300 is one example of a suitable device or system and is not intended to suggest any limitation as to the scope of use or functionality of the various embodiments described.

Other well-known computer systems, environments and/or configurations that may be suitable for use with the embodiments include, but are not limited to personal computers, server computers, hand-held or laptop devices, multiprocessor systems, microprocessor-based systems, set top boxes, game con soles, programmable consumer electronics, network PCs, minicomputers, mainframe computers, distributed computing environments that include any of the above systems or devices, and/or the like. Some or all of the components of computer system 1300 may be implemented in a cloud computing environment, such that resources and/or services may be made available via a computer network for selective use by the user devices.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability or configuration of the disclosure. Rather, the present description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the present description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1. A method comprising:
availing, to a user device associated with a user, an interface that includes one or more sets of queries and a set of components to receive a set of responses corresponding to the sets of queries;
receiving, from the user device, a particular set of responses corresponding to a particular set of queries;
leveraging one or more artificial intelligence techniques to process the particular set of responses to generate one or more metrics, wherein each of at least one of the one or more metrics is based on:
an extent to which responses provided by the user in a given session with the interface are consistent with each other;
a complexity or sophistication of responses provided by the user in the given session with the interface;
a degree to which responses provided by the user in the given session with the interface accord with grammatical rules and/or proper spelling;
an amount of time that the user spent providing responses during the given session; or
a number of times or cumulative amount of time that the user paused while providing responses during the given session;
determining, based on the one or more metrics, whether a condition is satisfied; and
in response to determining that the condition is satisfied, triggering a presentation, transmission or action that corresponds to an alert about a potential neurotoxicity or a preventative measure to reduce a likelihood of further neurotoxicity.

2. The method of claim 1, wherein each of the at least one of the one or more metrics is based on:
the degree to which each of the one or more metrics differ from a corresponding metric associated with one or more prior sessions of the user.

3. The method of claim 1, further comprising:
generating a composite score by aggregating each of two or more of the metrics for the given session.

4. The method of claim 1, further including:
preprocessing each response of the particular set of responses by generating a corresponding one or more tokens associated with each response.

5. The method of claim 1, wherein the generation of one or more metrics based on the extent to which the responses of the particular set of responses are consistent is estimated by:
generating a first distribution of positions and a second distribution of positions by assigning, each token of one or more tokens associated with each response of the particular set of responses, a position in a multi-dimensional space;
performing a comparison of the first distribution of positions relative to the second distribution of positions; and
calculating a metric of the one or more metrics estimating the consistency based on the comparison.

6. The method of claim 1, wherein the complexity or sophistication of the responses is estimated by leveraging a large language model (LLM).

7. The method of claim 1, wherein the condition includes negligible neurotoxicity, non-severe neurotoxicity or severe neurotoxicity.

8. A system comprising:
one or more data processors; and
a non-transitory computer readable storage medium containing instructions which, when executed on the one or more data processors, cause the one or more data processors to perform operations including:
avail, to a user device associated with a user, an interface that includes one or more sets of queries and a set of components to receive a set of responses corresponding to the sets of queries;
receive, from the user device, a particular set of responses corresponding to a particular set of queries;
leverage one or more artificial intelligence techniques to process the particular set of responses to generate one or more metrics, wherein each of at least one of the one or more metrics is based on:
an extent to which responses provided by the user in a given session with the interface are consistent with each other;
a complexity or sophistication of responses provided by the user in the given session with the interface;
a degree to which responses provided by the user in the given session with the interface accord with grammatical rules and/or proper spelling;
an amount of time that the user spent providing responses during the given session; or
a number of times or cumulative amount of time that the user paused while providing responses during the given session;
determine, based on the one or more metrics, whether a condition is satisfied; and
in response to determining that the condition is satisfied, triggering a presentation, transmission or action that corresponds to an alert about a potential neurotoxicity or a preventative measure to reduce a likelihood of further neurotoxicity.

9. The system of claim 8, wherein each of the at least one of the one or more metrics is based on:
the degree to which each of the one or more metrics differ from a corresponding metric associated with one or more prior sessions of the user.

10. The system of claim 8, further comprising:
generating a composite score by aggregating each of two or more of the metrics for the given session.

11. The system of claim 8, further including:
preprocessing each response of the particular set of responses by generating a corresponding one or more tokens associated with each response.

12. The system of claim 8, wherein the generation of one or more metrics based on the extent to which the responses of the particular set of responses are consistent is estimated by:
generating a first distribution of positions and a second distribution of positions by assigning, each token of one or more tokens associated with each response of the particular set of responses, a position in a multi-dimensional space;
performing a comparison of the first distribution of positions relative to the second distribution of positions; and
calculating a metric of the one or more metric estimating the consistency based on the comparison.

13. The system of claim 8, wherein the complexity or sophistication of the responses is estimated by leveraging a large language model (LLM).

14. The system of claim 8, wherein the condition includes negligible neurotoxicity, non-severe neurotoxicity or severe neurotoxicity.

15. A computer program product tangibly embodied in a non-transitory machine-readable storage medium, including instructions configured to cause one or more data processors to perform operations including:
availing, to a user device associated with a user, an interface that includes one or more sets of queries and a set of components to receive a set of responses corresponding to the sets of queries;
receiving, from the user device, a particular set of responses corresponding to a particular set of queries;
leveraging one or more artificial intelligence techniques to process the particular set of responses to generate one or more metrics, wherein each of at least one of the one or more metrics is based on:
an extent to which responses provided by the user in a given session with the interface are consistent with each other;
a complexity or sophistication of responses provided by the user in the given session with the interface;
a degree to which responses provided by the user in the given session with the interface accord with grammatical rules and/or proper spelling;
an amount of time that the user spent providing responses during the given session; or
a number of times or cumulative amount of time that the user paused while providing responses during the given session;
determining, based on the one or more metrics, whether a condition is satisfied; and
in response to determining that the condition is satisfied, triggering a presentation, transmission or action that corresponds to an alert about a potential neurotoxicity or a preventative measure to reduce a likelihood of further neurotoxicity.

16. The computer program product of claim 15, wherein each of the at least one of the one or more metrics is based on:
the degree to which each of the one or more metrics differ from a corresponding metric associated with one or more prior sessions of the user.

17. The computer program product of claim 15, further comprising:
generating a composite score by aggregating each of two or more of the metrics for the given session.

18. The computer program product of claim 15, further including:
preprocessing each response of the particular set of responses by generating a corresponding one or more tokens associated with each response.

19. The computer program product of claim 15, wherein the generation of one or more metrics based on the extent to which the responses of the particular set of responses are consistent is estimated by:
generating a first distribution of positions and a second distribution of positions by assigning, each token of one or more tokens associated with each response of the particular set of responses, a position in a multi-dimensional space;
performing a comparison of the first distribution of positions relative to the second distribution of positions; and
calculating a metric of the one or more metrics estimating the consistency based on the comparison.

20. The computer program product of claim 15, wherein the complexity or sophistication of the responses is estimated by leveraging a large language model (LLM).
